(19) United States
(12) Patent Application Publication
(10) Pub. No.: US 2017/0042725 A1
(43) Pub. Date: Feb. 16, 2017

(54) ORAL MOTION PRESERVATION DEVICE

(71) Applicant: SILVERFOX DENTAL & ORTHO, LLC, Sparks, MD (US)

(72) Inventor: Brian Francis SMITH, Baldwin, MD (US)

(21) Appl. No.: 15/233,138

(22) Filed: Aug. 10, 2016

Related U.S. Application Data
(60) Provisional application No. 62/204,234, filed on Aug. 12, 2015.

(51) Int. Cl. A61F 5/56; A61C 7/36

(52) U.S. Cl. CPC A61F 5/56; A61C 7/36; A61F 5/566; A61F 2005/563

(57) ABSTRACT

A device for the treatment of bruxism may include an articulation plate worn on one of the upper or lower jaw and a bite plate worn on the other of the upper or lower jaw, coupled by posts extending from one of the bite plate or articulation plate into housings in the other of the bite plate or articulation plate. First articulating components on one of the bite plate or articulation plate may engage second articulating components on the other of the bite plate or articulation plate to allow for 360 degree articulating motion. Removable bite pads between the bite plate and the teeth received in the bite plate, and/or between the articulation plate and the teeth received in the articulation plate may provide cushioning and impact absorption, and may be removed and replaced to promote good oral hygiene and avoid excessive wear.

### CROSS-REFERENCE TO RELATED APPLICATION(S)

This application claims priority to provisional Application Ser. No. 62/204,234, filed on Aug. 12, 2015, the entirety of which is incorporated herein by reference.

### FIELD

This document relates, generally, to an oral device, and in particular, to an oral device for the preservation of natural mandibular motion and prevention and/or reduction of bruxism.

### BACKGROUND

Bruxism generally refers to the excessive grinding and/or gnashing of the teeth, in which the top and bottom teeth forcefully slide against each other, and/or to the excessive clenching of the jaw. Frequent, severe and/or sustained bruxism may lead to Temporomandibular Joint Syndrome (TMJS) and/or Temporomandibular Joint Disorder (TMJD). Bruxism is an oral para-functional activity, in that it is not typically a conscious action, and not related to normal oral functions such as, for example, eating or talking. Symptoms such as facial pain, neck pain, hypersensitive teeth, aching jaw muscles, headaches and other such symptoms may be associated with bruxism. Bruxism may also cause tooth wear, bite misalignment, and damage and/or breakage of teeth and dental restorations such as crowns and fillings. A device that preserves natural jaw motion while providing a barrier to the underlying teeth, may be effective dissipating forces generated due to grinding and preserving natural motion, while separation of the upper an lower jaws may minimize clenching and related complications and/or symptoms.

### SUMMARY

In one aspect, an oral device may include a first plate defining a first channel configured to receive teeth of one of an upper jaw or a lower jaw, a second plate defining a second channel configured to receive teeth of the other of the upper jaw or the lower jaw, a coupling device configured to couple the first plate and the second plate, an articulating coupler configured to allow for articulation between the first plate and the second plate coupled by the coupling device, and a plurality of bite pads configured to be removably coupled in at least one of the first channel defined in the first plate or the second channel defined in the second plate.

In another aspect, an oral device may include a bite plate configured to receive teeth of one of an upper jaw or a lower jaw therein, an articulation plate configured to receive teeth of the other of the upper jaw or the lower jaw therein, a coupling device configured to couple the articulation plate and the bite plate, an articulation coupler configured to allow for articulation between the bite plate and the articulation plate coupled by the coupling device, and a plurality of bite pads configured to be disposed on an interior facing surface of the bite plate, between the bite plate and the teeth to be received therein, and on an interior facing surface of the articulation plate, between the articulation plate and the teeth to be received therein.

In some embodiments, an oral device may include a first plate configured to receive teeth of an upper jaw there, a second plate configured to receive teeth of a lower jaw therein, a plurality of bite pads removably coupled to at least one of the first plate or the second plate, and positioned between the respective plate and teeth received therein.

The details of one or more implementations are set forth in the accompanying drawings and the description below. Other features will be apparent from the description and drawings, and from the claims.

### BRIEF DESCRIPTION OF THE DRAWINGS

FIG. 1A is a disassembled perspective view of an exemplary bruxism device, in accordance with implementations described herein.

FIG. 1B is a perspective view of an inner side of the first plate of the exemplary bruxism device shown in FIG. 1A, in accordance with implementations described herein.

FIG. 1C is a perspective view of an inner side of a second plate of the exemplary bruxism device shown in FIG. 1A, in accordance with implementations described herein.

FIG. 2A is an assembled perspective view of the exemplary bruxism device shown in FIGS. 1A-1C, in accordance with implementations described herein.

FIG. 2B is a rear view of the exemplary bruxism device shown in FIG. 2A, in accordance with implementations described herein.

FIG. 2C is a cross sectional view of the exemplary bruxism device taken along line C-C of FIG. 2A, illustrating a coupling of a first articulating component and a second articulating component of the exemplary bruxism device, in accordance with implementations described herein.

FIG. 2D is a cross sectional view of the exemplary bruxism device taken along line D-D of FIG. 2A, illustrating a coupling of a post and a housing of the exemplary bruxism device, in accordance with implementations described herein.

FIGS. 3A-3C are front assembled views of a bruxism device, in accordance with implementations described herein.

FIGS. 4A-4C are disassembled perspective views of a bruxism device, in accordance with implementations described herein.

FIGS. 5A-5D illustrate bruxism devices worn by a user, in accordance with implementations described herein.

FIG. 6A is a disassembled perspective view of an exemplary oral device, in accordance with implementations described herein.

FIG. 6B is a perspective view of an inner side of the first plate of the exemplary oral device shown in FIG. 6A, in accordance with implementations described herein.

FIG. 6C is a perspective view of an inner side of a second plate of the exemplary oral device shown in FIG. 6A, in accordance with implementations described herein.

FIGS. 6D and 6E are perspective views of an inner side of a first plate and/or a second plate of the exemplary oral device shown in FIG. 6A, in accordance with implementations described herein.

FIGS. 7A-7D illustrate an example of bite pads included on a plate of the exemplary oral device shown in FIGS. 6A-6E, in accordance with implementations described herein.

FIGS. 8A-8D are rear views of an end of a plate of the exemplary oral device shown in FIGS. 6A-6E, illustrating couplings of a bite pad to the plate, in accordance with implementations described herein.

FIG. 9 illustrates the exemplary oral device shown in FIGS. 6A-6E, worn by a user, in accordance with implementations described herein.

### DETAILED DESCRIPTION

The following detailed description sets forth various exemplary implementations of a bruxism device. The description is not to be taken in a limiting sense, but is made merely for the purpose of illustrating the general principles as broadly described herein.

An oral bruxism device, in accordance with implementations described herein, may preserve natural motion of the upper and lower jaws of a patient in the event of grinding and/or gnashing and/or clenching of the jaws. In some implementations, an oral bruxism device may include a static bite plate positioned between the upper jaw and the lower jaw, and in particular, between the teeth of the upper jaw and the teeth of the lower jaw. This type of static bite plate may provide some protection against wear of the teeth of the upper and/or lower jaws. However, this type of static bite plate may still provide some resistance against the natural movement of the jaws, and thus may still transfer deleterious stress forces generated by a grinding motion onto the underlying teeth as well as the temporomandibular joints and connective soft tissues.

A bruxism device, in accordance with implementations described herein, may allow for the natural movement of the jaws and dissipate the stress forces generated by the grinding motion. A bruxism device, in accordance with implementations described herein, may include, for example, a bite plate connected to an articulation plate. The bite plate may include a plurality of first articulating components extending generally perpendicularly from the bite plate. The articulation plate may include an inner material capable of being fit or conformed to the upper jaw or the lower jaw, or conformed to fit a form-fitted, or custom-fitted, dental/oral tray or other type of dental/oral mouth guard. The articulation plate may also include a plurality of second articulating components adapted so that each second articulating component may articulately receive a single one of the plurality of first articulating components included on the bite plate. The bruxism device may be mounted on the upper jaw or the lower jaw. A separation distance between the bite plate and the articulation plate may be adjusted by a damper, so that corresponding teeth may be separated by a predetermined distance from the bruxism device. The damper may be an articulating damper, or a non-articulating damper. When a user wearing the bruxism device grinds his/her teeth, the articulating components may provide a ‘gliding’ affect for the upper and/or lower jaws, so that the teeth do not encounter significant friction and the jaws do not encounter significant resistance. The non-constrained motion of the upper and lower jaws provided for by a bruxism device in accordance with implementations described herein may allow the jaws to maintain a substantially natural motion state.

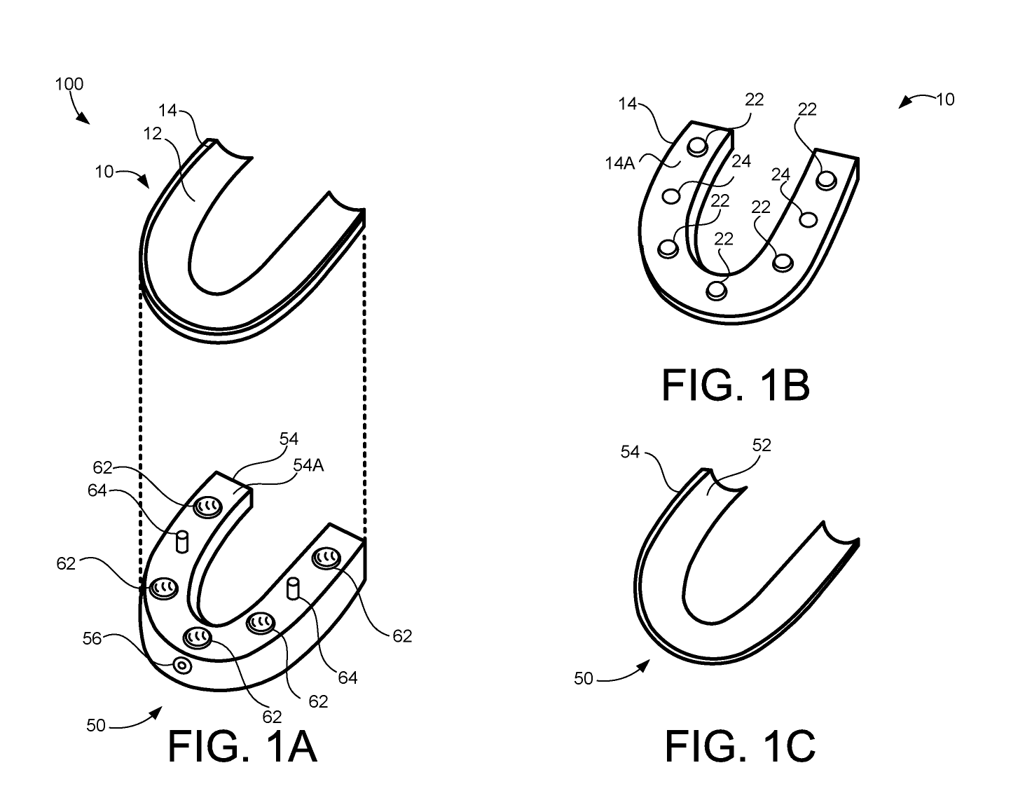

An example implementation of a bruxism device is shown in the disassembled perspective view of FIG. 1A. The bruxism device 100 may include, for example, a first plate 10 selectively coupled to a second plate 50. For ease of discussion, the first plate may hereinafter be referred to as a bite plate 10, and the second plate may hereinafter be referred to as an articulation plate 50.

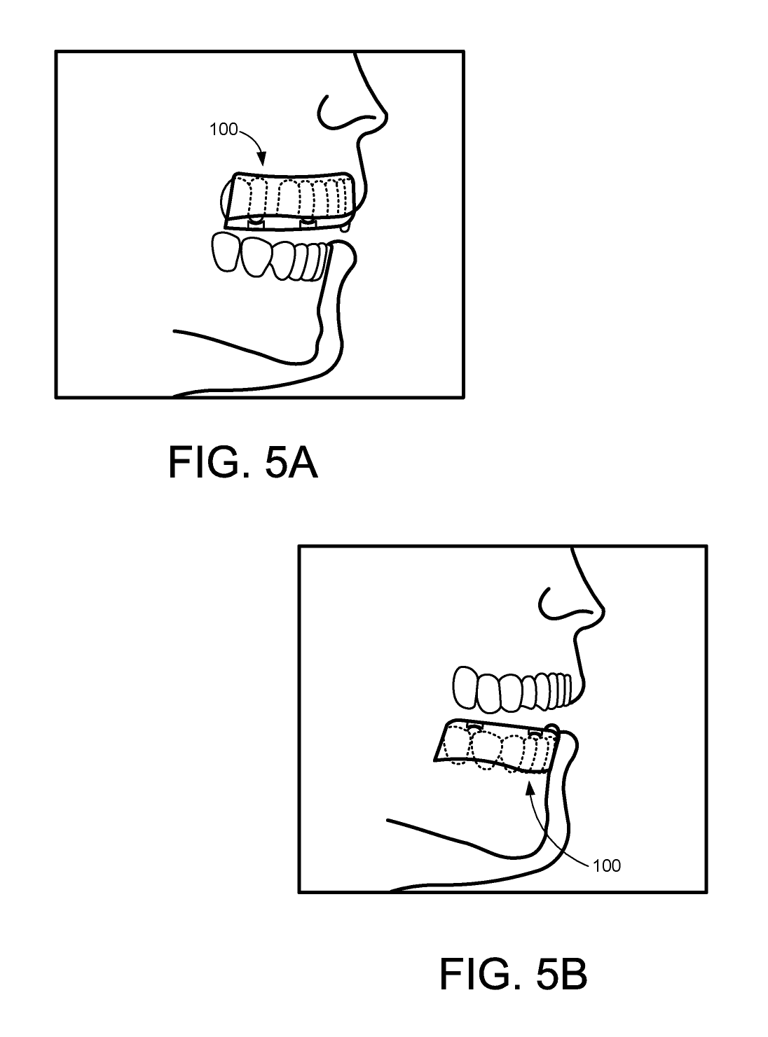

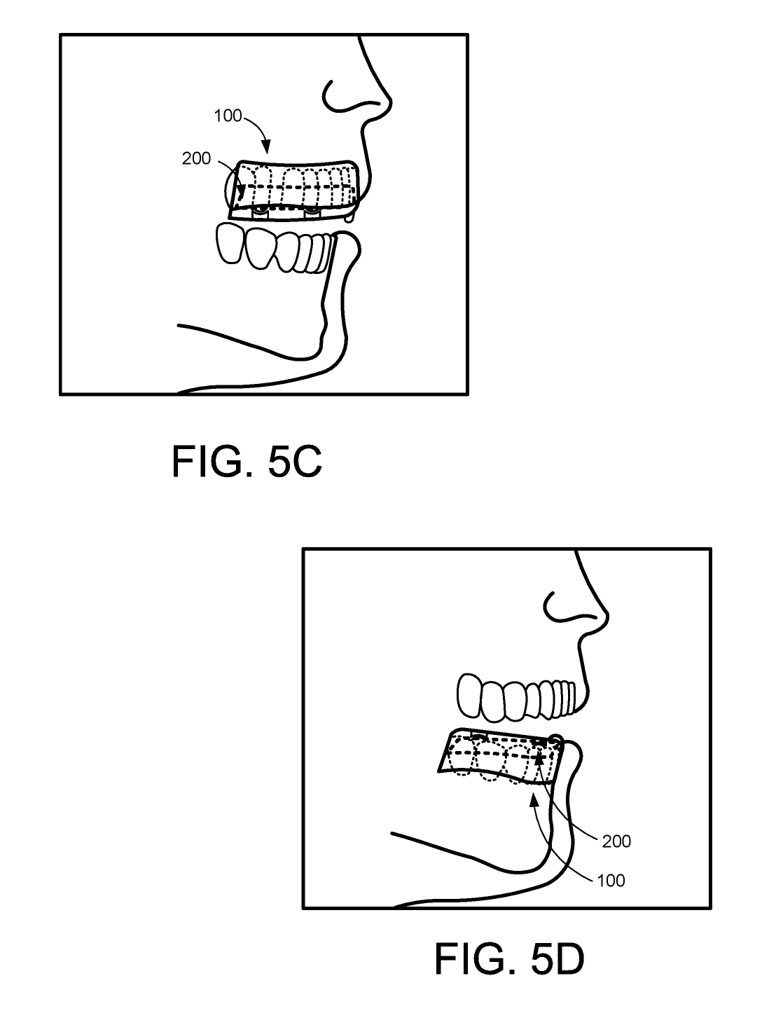

In some implementations, the bite plate 10 may include a soft inner lining 12 and a rigid outer lining 14, the inner lining 12 being situated on an inner side of the bite plate 10 in which the teeth of one of the upper jaw or the lower jaw are to be received, and the outer lining 14 defining an outer side of the bite plate 10, facing the articulation plate 50. In some implementations, the inner lining 12 may be made of a relatively soft, relatively resilient thermoplastic material such as, but not limited to, plastic. Properties of the material of the inner lining 12 may allow the inner lining 12 to conform to the teeth of a user while also cushioning the jaw against impact and teeth-gnashing forces. In some implementations, the inner lining 12 may conform to, or receive, or be fitted over an oral device 200 (see, for example, FIGS. 5C and 5D), such as, for example a pre-existing oral device 200 which the user has positioned on the teeth, such as, for example, whitening trays and the like, which may have been previously customized for a particular user. In some implementations, the outer lining 14 of the bite plate 10 may be made of a relatively rigid, somewhat resilient thermoplastic material, such as, but not limited to, plastic. The inner lining 12 may be secured to the outer lining 14 by, for example, an adhesive, a lamination process, and the like. In some implementations, as shown in FIG. 1B, a plurality of first articulating components 22 may extend outward from a surface 14a of the outer lining 14 facing the articulation plate 50, and may extend in a direction that is substantially perpendicular to the surface 14a of the outer lining 14. In some embodiments, a plurality of housings 24, or openings 24, or recesses 24, may also be defined in the surface 14a of the outer lining 14.

The articulation plate 50 may be positioned opposite, or facing, the bite plate 10, and may be coupled to the bite plate 10. The articulation plate 50 may include a soft inner lining 52, as shown in FIG. 1C, and a rigid outer lining 54, the inner lining 52 being situated on an inner side of the articulation plate 50 in which the teeth of the other of the upper jaw or the lower jaw are to be received, and the outer lining 54 defining an outer side of the articulation plate 50, facing the bite plate 10. In some implementations, the inner lining 52 may be made of a relatively soft, relatively resilient thermoplastic material such as, but not limited to, plastic. Properties of the material of the inner lining 52 may allow the inner lining 52 to conform to the teeth while also cushioning the jaw against impact and teeth-gnashing forces. In some embodiments, the inner lining 52 may conform to, or receive, or be fitted over another oral device 200 as described above, which the user has positioned on the teeth, and which may have been previously customized for a particular user, such as, for example, whitening trays and the like. In some implementations, the outer lining 54 may be made of a relatively rigid, somewhat resilient thermoplastic material, such as, but not limited to, plastic. The inner lining 52 may be secured to the outer lining 54 by, for example, an adhesive, a lamination process, and the like. A plurality of second articulating components 62 and a plurality of posts 64 may be defined on a surface 54a of the outer lining 54 of the articulation plate 50, facing the bite plate 10.

When the bite plate 10 and the articulation plate 50 are coupled, the surface 54a of the articulation plate 50 on which the plurality of second articulating components 62 and the plurality of posts 64 are located may be positioned opposite, or facing, the surface 14a of the outer liner 14 of the bite plate 10 on which the plurality of first articulating components 22 and the plurality of housings 24 are located. The plurality of second articulating components 62 may be engaged with the plurality first articulating components 22 provided on the surface 14a of the bite plate 10. The plurality of posts 64 may be engaged with the plurality of housings 24 defined in the surface 14a of the bite plate 10. The bite plate 10 may interface with the teeth of one of the upper jaw or the lower jaw, and the articulation plate 50 may interface with the teeth of the other of the upper jaw or the lower jaw so as to minimize the transfer of teeth-gnashing forces.

Each housing 24 formed in the surface 14a of the bite plate 10 may be adapted to receive a single, corresponding post 64 of the articulation plate 50. When the posts 64 are received in their respective housings 24, the bite plate 10 and the articulation plate 50 may be coupled. In some implementations, each post 64 may be fixed in its respective housing 24 by, for example, an adhesive or other attaching material. In some implementations, each post 64 may be fixed in its respective housing 24 such that a gap is defined between the surface 14a of the bite plate 10 and the surface 54a of the articulation plate 50.

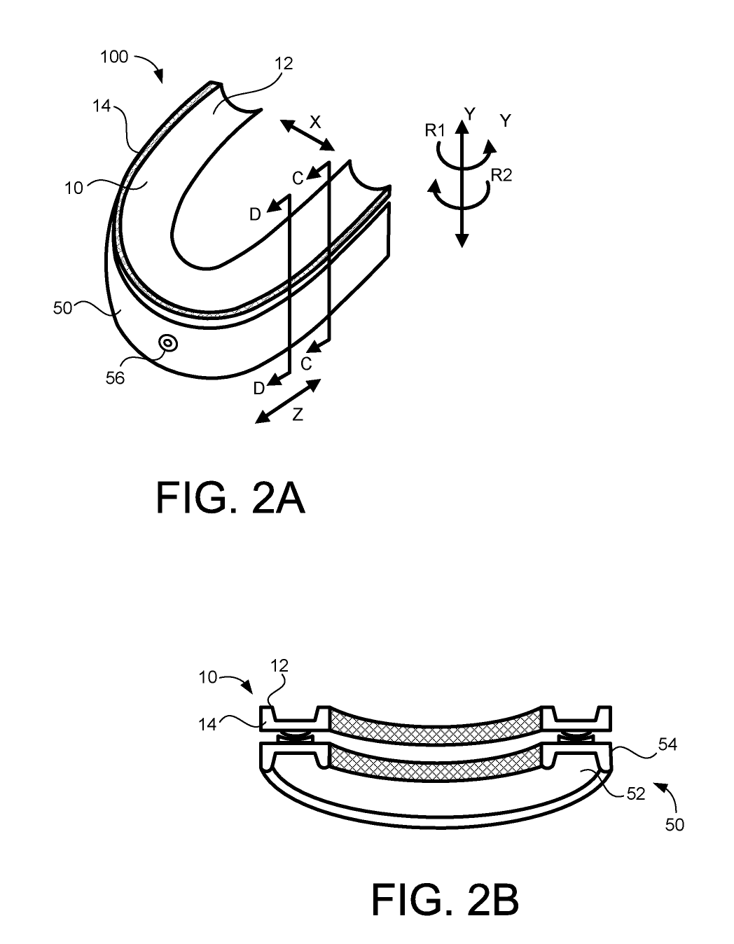

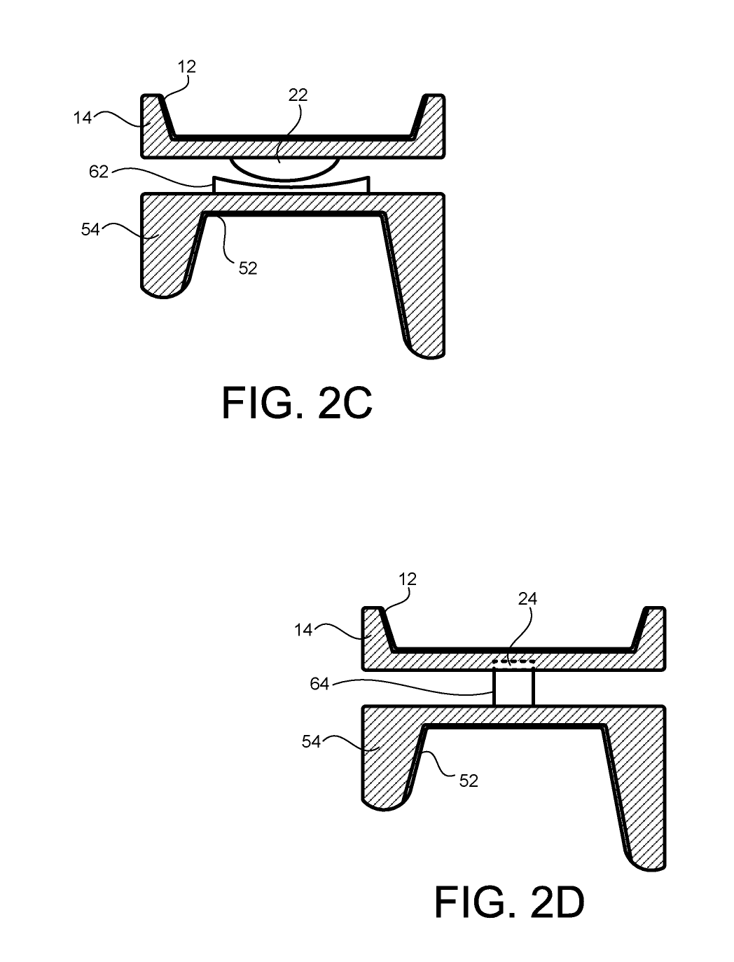

In the illustrated implementations, the posts 64 formed on the example articulation plate 50 shown in FIG. 1A have a substantially circular cross section, and extend outward, in a substantially perpendicular direction from the surface 54a of the articulation plate 50 in a substantially cylindrical form. The housings 24, which are formed as recesses in the example bite plate 10 shown in FIG. 1B, also have a substantially circular cross section, extending into the outer lining 14 of the bite plate 10 in a substantially cylindrical form, so as to correspond to the posts 64 and allow the posts 64 to be received and secured in the housings 24. However, in some implementations, the posts 64 and housings 24 may have other cross sectional shapes. For example, in some implementations, the posts 64 and housings 24 may have, for example, an elliptical cross section, a square cross section, a rectangular, cross section, or other polygonal cross sectional shape. In some embodiments, a cross sectional shape of the housing 24 corresponds to a cross sectional shape of the post 64 to facilitate insertion and securing of the post 64 in the housing 24. Insertion of one of the posts 64 of the articulating plate 50 into a corresponding housing 24 formed in the bite plate 10 is shown in FIG. 2D.

In some implementations, the posts 64 may be made of a flexible, somewhat deformable material. Flexibility of the posts 64 fixed in the housings 24 may allow for some movement, such as, for example, longitudinal movement between the bite plate 10 and the articulation plate 50 (in a front and rear direction of the jaws, as shown by the arrow Z in FIG. 2A), and/or transverse movement between the bite plate 10 and the articulation plate 50 (in a right and left direction of the jaws, as shown by the arrow X in FIG. 2A), some compressive movement between the bite plate 10 and the articulation plate 50 (as shown by the arrow Y in FIG. 2A), and/or a combination of the longitudinal and transverse movement which may produce a rotational movement (as shown by the arrows R1 and R2 in FIG. 2A). Flexibility of the posts 64 fixed in the housings 24 and the resulting flexibility in the X, Y, Z, R1 and R2 directions as shown may provide for both shock absorption and stability between the bite plate 10 and the articulation plate 50.

In some implementations, while being flexible, the posts 64 may also be substantially solid, and relatively free of openings that may otherwise entrap debris and/or moisture, and relatively tightly fitted in the housings to avoid entrapment of debris and/or moisture in spaces between the posts 64 and the housings 24.

As shown in FIG. 2C, each second articulating component 62 may be adapted to articulately receive, and/or engage with, a single, corresponding first articulating component 22. In some implementations, the plurality of first articulating components 22 and plurality of second articulating components 62, may, in a received or engaged arrangement, be referred to together as an articulating connector.

In the example implementation shown in FIGS. 1A-1B and 2C, each of the plurality of first articulating components 22 is defined by a protrusion that extends outward, from the mating surface 14a of the bite plate 10 and having a substantially semispherical shape or surface 22a. In the example implementation shown in FIGS. 1A-1B and 2C, each of the plurality of second articulating components 62 includes a receiving surface 62a located on the mating surface 54a of the articulation plate 50, at positions corresponding to the positions of the plurality of first articulating components 22. The receiving surface 62a of each of the second articulating components may have a curved, or arcuate, contour, that facilitates essentially 360 degree movement of the semispherical surface 22a of the first articulating component 22 received therein. The curved indentation, or curved recess, defined by the receiving surface 62a allows the first articulating component 22 to be received in and move freely within the second articulating component 62 without becoming easily disengaged.

In some implementations, the first and second articulating components 22 and 62 may include, for example, ball and trough components, ball-in-socket components, two plates with a mobile/sliding core positioned between the two plates, and other such components that, with the articulating components in an aligned and engaged arrangement, would provide for an articulating relationship between the bite plate 10 and the articulation plate 50.

In the views shown in FIGS. 1A, 2A-2B and 3A-3B, the bruxism device 100 is oriented with the bite plate 10 at an upper portion of the device 100 and the articulation plate 50 at a lower portion of the device 100. When the bite plate 10 and the articulation plate 50 are oriented in this manner, and coupled in the manner shown in FIGS. 2A-2B, the device 100 may be worn so that the teeth of the upper jaw are received in the inner lining 12 of the bite plate 10, and the teeth of the lower jaw are received in the inner lining 52 of the articulation plate 50, such that the device 100 is essentially “worn” or “mounted” on the upper jaw. The bite plate 10 and articulation plate 50 of the bruxism device 100 are oriented as shown in FIGS. 1A, 2A and 2B simply to facilitate description of the features of the device 100. In some implementations, the device 100 may be worn in a position inverted from what is shown in FIGS. 1A, 2A-2B and 3A-3B. That is, in some embodiments, the teeth of the bottom jaw may be received in the inner lining 12 of the bite plate 10, and the teeth of the upper jaw may be received in the inner lining 52 of the articulation plate 50, such that the device 100 is essentially “worn” or “mounted” on the lower jaw.

In the example implementation shown in FIGS. 1A-1C, 2A-2D and 3A-3B, the first articulating components 22 and the housings 24 are located on the surface 14a of the bite plate 10, and the second articulating components 62 and the posts 64 are located on the surface 54a of the articulating plate 50. However, in some implementations, the first articulating components 22 and the housings 24 may be located on the surface 54a of the articulation plate 50, and the second articulating components 62 and the posts 64 may be located on the surface 14a of the bite plate 10. Other various arrangements of the first and second articulating components 22 and 62, the housings 24 and the posts 64 on the surfaces 14a and 54a of the bite plate 10 and the articulating plate 50 may also be implemented, as long as engagement between the first and second articulating components 22 and 62, and between the posts 64 and housings 24, may be maintained. For example, while the illustrated implementation includes the “male” articulating component 22 on the bite plate 10 and the “female” articulating component 62 on the articulation plate 50, respective installation of the articulating components 22 and 62 on the bite plate 10 and the articulation plate 50 may be reversed, for some, or for all, of the pairs of articulating components 22/62. Similarly, while the illustrated implementation includes the “male” post 64 on the articulation plate 50 and the “female” housing 24 in the bite plate 10, respective installation of the housings 24 and posts 64 on the bite plate 10 and the articulation plate 50 may be reversed, for some, or for all, of the pairs of housings 24 and posts 64.

In the illustrated implementation shown in FIGS. 1A-1C, the bite plate 10 and articulation plate 50 includes two pairs of first and second articulating components 22 and 62 arranged on a first lateral side (for example, a left lateral side) of the bruxism device 100, two pairs of first and second articulating components 22 and 62 arranged on a second lateral side (for example, a right lateral side) of the bruxism device 100, and one pair of first and second articulating components 22 and 62 arranged at a front central portion of the bruxism device 100, or five total pairs of first and second articulating components 22 and 62 movably coupling the bite plate 10 and the articulation plate 50. However, in some implementations, more, or fewer, pairs of first and second articulating components 22 and 62 may be arranged along the mating surfaces of the bite plate 10 and the articulation plate 50, depending, for example, on a size of the bruxism device, and amount of correction and/or compensation required, and other such factors.

In some implementations, an articulating damper 56 may be provided in, for example, the articulating plate 50. The articulating damper 56 may include a mechanism that adjusts and maintains a desired position between the bite plate 10 and the articulation plate 50, and thus a desired amount of separation between the corresponding teeth of the upper jaw and the lower jaw. In some implementations, the articulating damper 56 may be included at a front portion of the articulation plate 50, so that the articulating damper 56 is easily accessible and may provide for adjustment of the separation distance between the front teeth, which may facilitate the further dissipation of grinding and clenching forces. In some embodiments, this separation distance may be between approximately 0.0 mm and 50.0 mm, depending, for example, on the size of a particular bruxism device and/or the individual needs and/or comfort of a particular user.

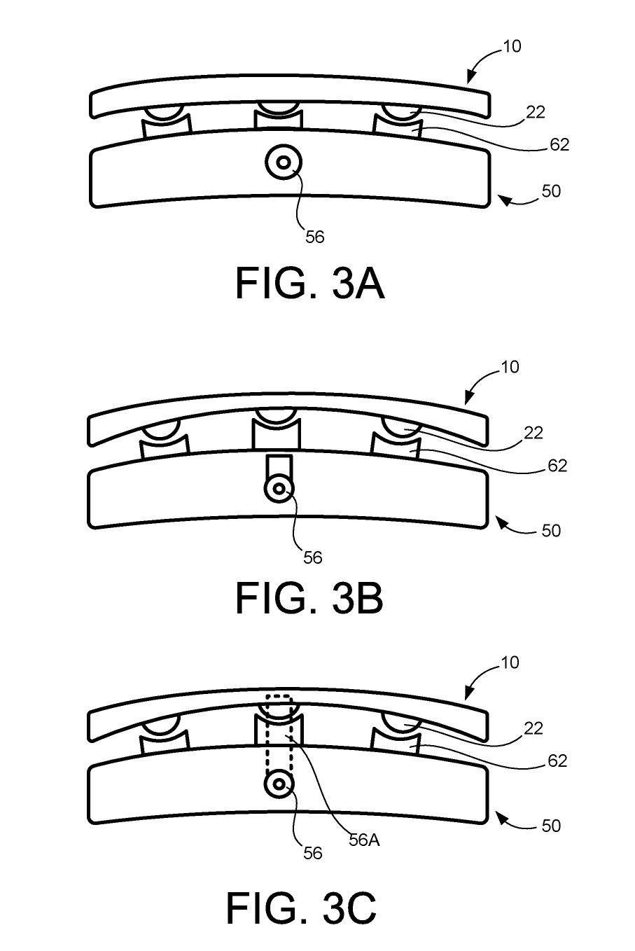

FIG. 3A is a front view of the exemplary bruxism device 100, in which the articulating damper 56 has not been actuated. In the example shown in FIG. 3B, the articulating damper 56 is provided at a front, or anterior, portion of the device 100, and has been actuated to increase a separation distance, or damper opening, between the front teeth received in the bite plate 10 and the front teeth received in the articulation plate 50. The articulating damper 56 may employ, for example, a cam, a spring, a piston, a jack, a wedge and the like to generate and maintain the separation distance, and may be adjusted by, for example, a knob, a hex key, a button and the like. In some embodiments, the articulating damper 56 may exert a force on an underside of the outer lining 54, pushing the surface 54a in the direction of the surface 14a, and consequently increasing a separation distance between the teeth received in the articulation plate 50 and the bite plate 10 in the general area of the damper 56. In some implementations, this force exerted by the articulating damper 56 may be exerted on a corresponding one of the second articulating components 62. In some implementations, actuation may cause a portion of the damper 56 to extend up through the surface 54a of the articulation plate 50 to push on the surface 14a of the bite plate 10.

As shown in FIG. 3C, in some implementations, the damper 56 may be coupled to a riser 56A. In response to actuation of the damper 56, the riser 56A may protrude through an opening in the articulation plate 50 and an opening in the bite plate 10, causing the riser 56A to directly contact the teeth received in the bite plate 10 to maintain a separation distance between the bite plate 10 and the teeth.

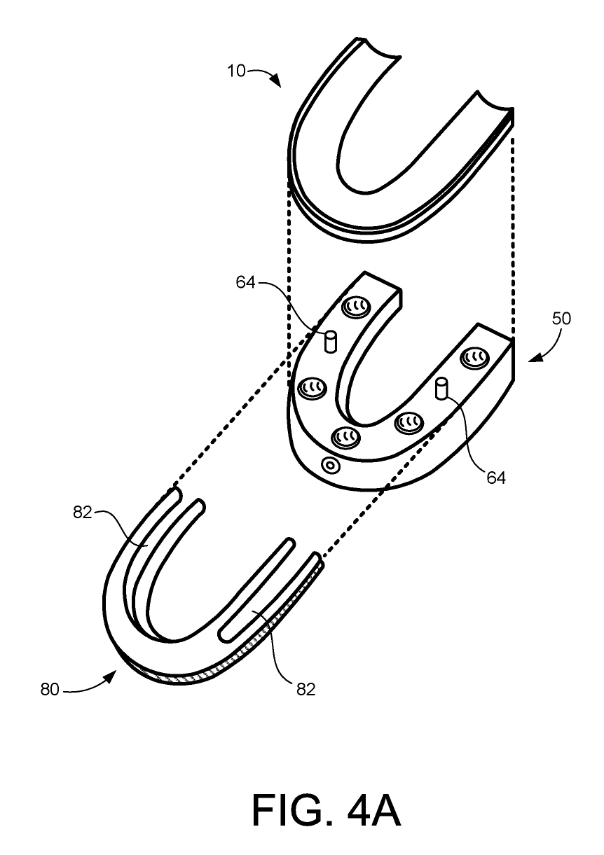

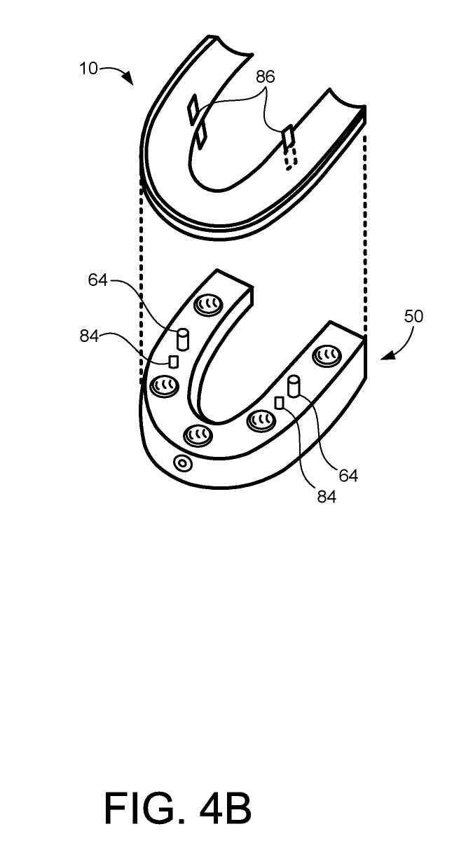

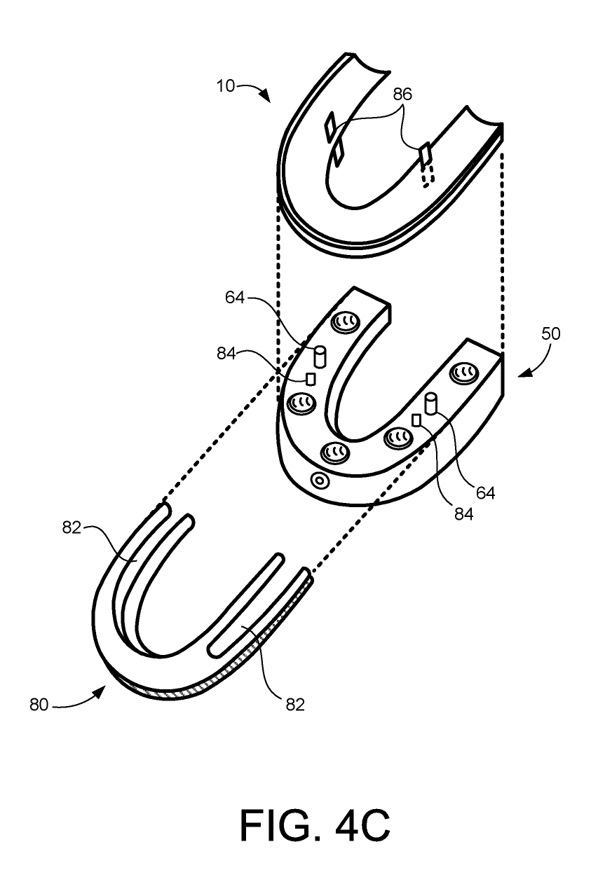

In some implementations, the bite plate 10 and the articulation plate 50 may be secured in a desired position to limit relative movement of the jaws in a particular direction, or to limit relative movement of the jaws to a particular amount. For example, as shown in FIG. 4A, in some implementations, a locking wafer 80 may be inserted between the bite plate 10 and the articulation plate 50 to secure, or lock, a relative position of the bite plate 10 and the articulation plate 50. The locking wafer 80 may include channels 82 extending longitudinally along opposite side portions of the locking wafer 80. The locking wafer 80 may slide into place on the articulation plate 50, as illustrated by the dotted lines shown in FIG. 4A, with the posts 64 on opposite sides of the articulation plate 50 sliding along the channels 82 on the opposite sides of the locking wafer 80 to guide the locking wafer 80 into position on the articulation plate 50. The locking wafer 80 may remain secured in its position between the bite plate 10 and the articulation plate 50 due to some amount of friction between the locking wafer 80 and the bite plate 10 and/or articulation plate 50, as well as the positioning of the posts 64 in the channels 82.

In some implementations, a plurality of first locking pins 84 and a plurality of second locking pins 86 may secure, or lock, a relative position of the bite plate 10 and the articulation plate 50, as shown in FIG. 4B. In some implementations, the plurality of first locking pins 84 and/or the plurality of second locking pins 86 may be removable from and insertable into corresponding openings in the articulation plate 50 and the bite plate 10, respectively, so that a desired position of the bite plate 10 and the articulation plate 50 may be selected and secured, and adjusted as necessary. In some implementations, the plurality of first locking pins 84 and/or the plurality of second locking pins 86 may be, for example, tension mounted so that pressure applied to the first locking pins 84 and/or the second locking pins 86 may cause the first locking pins 84 and/or the second locking pins 86 to be retracted and/or extended.

In some implementations, the bruxism device 100 may include both the locking wafer 80 and the plurality of first locking pins 84 and/or the plurality of second locking pins 86, as shown in FIG. 4C. In this implementation, as the locking wafer 80 slides into place, the plurality of first locking pins 84 and/or the plurality of second locking pins 86 may extend from the respective bite plate 10 and/or articulation plate 50 for engagement with the locking wafer 80. In this manner, the locking wafer 80 may be positioned and secured between the articulation plate 50 and the bite plate 10 to establish and secure a predetermined position of the articulation plate 50 and the bite plate 10.

Securing, or locking, the articulation plate 50 and the bite plate 10 with either the locking wafer 80 as shown in FIG. 4A, the plurality of first locking pins 84 and/or the plurality of second locking pins 86 as shown in FIG. 4B, or both the locking wafer 80 and the locking pins 84 and/or 86 as shown in FIG. 4C, may cause the device to be in a static, non articulating state, with the bite plate 10 and the articulation plate 50 maintained in a desired relative position.

In use, as shown in FIG. 5A, the bruxism device 100 may be worn, or mounted, on the upper jaw, with the teeth of the lower jaw received in the inner lining 52 of the articulation plate 50 when the jaws are in a closed position. As shown in FIG. 5B, the bruxism device 100 may be worn, or mounted, on the lower jaw, with the teeth of the upper jaw received in the inner lining 12 of the bite plate 10 when the jaws are in the closed position. The bruxism device 100 may be available in a variety of sizes, such as, for example, small, medium, large, youth, adult and the like, so that the bruxism device 100 may be adapted to a vast majority of jaw sizes.

In use, as shown in FIG. 5C, the bruxism device 100 may be worn, or mounted, on the upper jaw, with a separate oral tray 200 positioned on the teeth of the lower jaw, and the separate oral tray 200 being received in, or fitted in, the inner lining 52 of the articulation plate 50 when the jaws are in a closed position. As shown in FIG. 5D, the bruxism device 100 may be worn, or mounted, on the lower jaw, with the separate oral tray 200 on the teeth of the upper jaw, and the separate oral tray 200 being received in, or fitted in, the inner lining 12 of the bite plate 10 when the jaws are in the closed position. As noted above, the bruxism device 100 may be available in a variety of sizes, to adapt to the vast majority of jaw sizes and/or to accommodate various different types of separate, customized trays which the user may already have on hand.

In some implementations, an impression of the relevant mouth cavity may be taken, and then one or both of the plates may be conformed to the impression. Thereafter, the articulating damper 56 may be actuated to adjust and set an amount of separation between the articulation plate 50 and the bite plate 10 that will yield the desired amount of separation between the corresponding teeth of the upper and lower jaws. The bruxism device 100 may then be mounted to either the upper jaw, as shown in FIG. 4A, or the lower jaw, as shown in FIG. 4B. In mounting the bruxism device 100 to the upper jaw, the teeth of the upper jaw may be received in, for example, the inner lining 52 of the articulation plate 50, so that, when the jaws are in the closed position, the teeth of the lower jaw are received in the inner lining 12 of the bite plate 10. Similarly, in mounting the bruxism device 100 to the lower jaw, the teeth of the lower jaw may be received in, for example, the inner lining 52 of the articulation plate 50, so that, when the jaws are in the closed position, the teeth of the upper jaw are received in the inner lining 12 of the bite plate 10.

In some implementations, the bruxism device 100 may be customized by the user so that the bite plate 10 and/or the articulation plate 50 may be custom fitted to the particular user's teeth and jaws. For example, in some embodiments, the bite plate 10 and/or the articulation plate 50 may be heated and then positioned in place on the user's teeth/jaws, so that the material of the inner lining 12 of the bite plate 10 and/or the material of the inner lining 52 of the articulation plate 50 may be molded to conform to the user's teeth and jaws. This customized, conformal shape of the inner lining 12 and/or the inner lining 52 may then set as the inner lining 12 and/or the inner lining 52 cools. In some embodiments, this type of customization may be done by the user, providing a less expensive and faster customized solution than a custom device made from impressions taken by a dental professional.

As noted above, in some implementations, the bite plate 10 and/or the articulation plate 50, and in particular the inner lining 12 of the bite plate 10 and/or the inner lining 52 of the articulation plate 50, may be adapted to receive another type of oral device and/or tray 200 that a user may already have on hand, such as, for example, night guard trays or whitening trays, orthodontic trays, and other such oral devices that may have been previously customized for the user which the user may wish to wear in conjunction with the bruxism device 100. In this instance, the customized tray(s) 200 may be worn on the teeth, and then the bruxism device 100 may be worn over the tray(s) 200. This may allow the user to benefit from previous customization of the tray(s) 200, continue the treatment intended by the tray(s) 200, and still reap the additional benefits provided by the bruxism device 100.

A bruxism device 100, in accordance with implementations described herein, may enable articulating movement of the upper and lower jaws in any number of different directions due to, for example, the essentially 360 degree motion supported by the plurality of first articulating components respectively engaged with the plurality of second articulating components, together with the flexibility and multiple degrees of freedom of motion supported by the plurality of flexible posts respectively received in the plurality of housings. Thus, when wearing the bruxism device 100, if clenching does occur, the jaw may articulate in a manner corresponding to its naturally intended movement and dissipate forces on the temporomandibular joint, with the distance between certain teeth of the upper and lower jaws spaced a predetermined distance apart due to the height adjustment provided by the articulating damper, rather than remaining in a static clenched state in which damage to the joints and connective tissue may occur. Moreover, when wearing the bruxism device 100, if grinding does occur, the jaw may experience a “gliding” effect due to the plurality of first and second articulating components, without encountering the friction and/or resistance normally encountered when grinding, maintaining the jaws in a natural motion state and avoiding the typical wear, pain and damage normally associated with grinding.

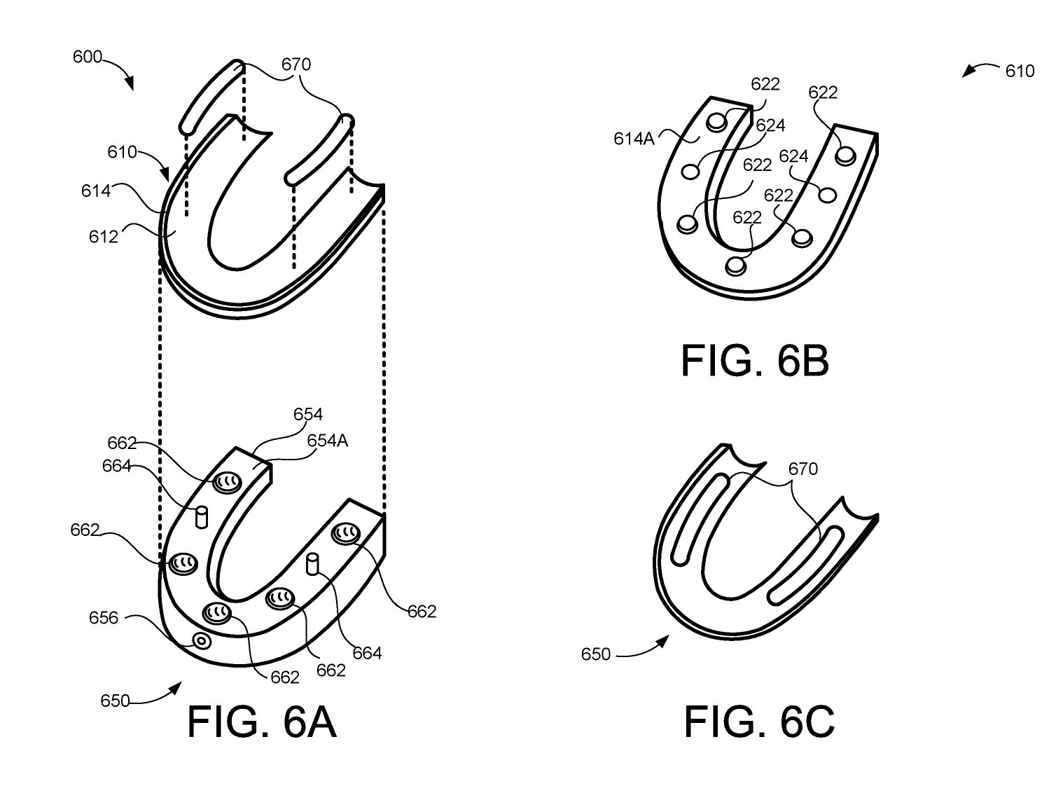

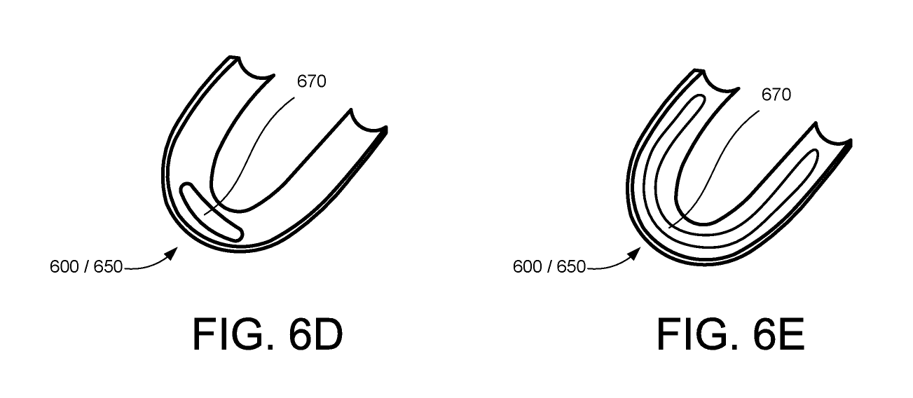

An example implementation of an oral device, in accordance with implementations described herein, is shown in the disassembled perspective view of FIG. 6A. In addition to, or instead of, the various example features described above, the example oral device shown in FIGS. 6A-6C may include removable and/or replaceable bite pads 670. The bite pads 670 may be fitted into a user's existing oral device, so that the bite pads 670 may be removed and replaced regularly within the existing oral device. In some implementations, the bite pads 670 may have various features which allow the user to customize the bite pads 670 for the user's particular oral device. For example, the bite pads 670 may vary in size, shape, hardness/softness, resilience, flavor, scent and the like, so that a user may select bite pads 670 for use in an existing oral device to suit specific preferences and oral/dental needs. In some implementations, used bite pads 670 may be regularly removed and replaced with new bite pads 670. Regular removal and replacement of bite pads 670 may help maintain good oral hygiene, may help maintain a healthy TMJ, may provide for safety during cleaning, may enhance user comfort as bite pads 670 wear, may enhance effectiveness of the oral device, and may allow a user to vary features of the oral device by varying the type of bite pads 670 used with the oral device. Bite pads 670 that are easily removable and/or replaceable by the user may also provide the user with a more economical approach to using, maintaining and customizing an oral device to specific needs, allowing the oral device to be used long term while replacing only the worn bite pads 670.

As shown in the disassembled perspective view of FIG. 6A, an oral device 600, in accordance with implementations described herein, may include, for example, a first plate 610 selectively coupled to a second plate 650. For ease of discussion, the first plate 610 may hereinafter be referred to as a bite plate 610, and the second plate may hereinafter be referred to as an articulation plate 650. The bite plate 610 may include an inner portion, the inner portion defining a channel in which the teeth of one of the upper jar or the lower jaw are to be received, and an outer portion defining an exterior facing portion of the bite plate 610. In some implementations, the bite plate 610 may include an inner lining 612 situated on an inner side of the bite plate 610, and an outer lining 614 defining an outer side of the bite plate 610, facing the articulation plate 650. In some implementations, the inner lining 612 may define the channel in which the teeth of one of the upper jaw or the lower jaw are to be received. The articulation plate 650 may include an inner portion, the inner portion defining a channel in which the teeth of one of the upper jar or the lower jaw are to be received, and an outer portion defining an exterior facing portion of the articulation plate 650. In some implementations, the articulation plate 650 may include an inner lining 652, as shown in FIG. 6C, situated on an inner side of the articulation plate 650, and an outer lining 654 defining an outer side of the articulation plate 650, facing the bite plate 610. In some implementations, the inner lining 652 may define the channel in which the teeth of the other of the upper jaw or the lower jaw are to be received.

In some implementations, as shown in FIG. 6B, a plurality of first articulating components 622 may extend outward from a surface 614a of the outer lining 614, and a plurality of housings 624, or openings 624, or recesses 624, may be defined in the surface 614a of the outer lining 614. In some implementations, as shown in FIG. 6A, a plurality of second articulating components 662 and a plurality of posts 664 may be defined on a surface 654a of the outer lining 654 of the articulation plate 650, facing the bite plate 610. Features of the first articulating components 622, second articulating components 662, housings 624 and posts 664, may be similar to the features described above with respect to FIGS. 1A through 1D. Additionally, the first articulating components 622 may engage with the second articulating components 662, and the posts 664 may engage with the housings 624, as described above with respect to FIGS. 1A through 1D. Thus, further detailed description of these features will not be repeated.

In the example implementation shown in FIGS. 6A-6C, the first articulating components 622 and the housings 624 are on the bite plate 610, and the second articulating components 662 and the posts 664 are on the articulation plate 650. However, in some implementations, respective positions of the first articulating components 622 on the bite plate 610 and the second articulating components 662 on the articulation plate 650 may be reversed, and/or respective positions of the housings 624 in the bite plate 610 and posts 664 on the articulation plate 650 may be reversed, and/or quantities of the first and second articulating components 622 and 624, posts 664 and housings 624 may be varied as necessary for a particular implementation.

In some implementations, the outer lining 614 of the bite plate 610 may be made of a relatively rigid, somewhat resilient thermoplastic material, such as, but not limited to, plastic. In some implementations, the inner lining 612 of the bite plate 610 may be made of the same material as the outer lining 614, or may be made of a different material than the outer lining 614, and may be secured to the outer lining 614 by, for example, an adhesive, a lamination process, and the like. In some embodiments, the outer lining 614 and the inner lining 612 of the bite plate 610 may be integrally formed. Similarly, in some implementations, the outer lining 654 of the articulation plate 650 may be made of a relatively rigid, somewhat resilient thermoplastic material, such as, but not limited to, plastic. In some implementations, the inner lining 652 of the articulation plate 650 may be made of the same material as the outer lining 654, or may be made of a different material than the outer lining 654, and may be secured to the outer lining 654 by, for example, an adhesive, a lamination process, and the like. In some embodiments, the outer lining 654 and the inner lining 652 of the articulation plate 650 may be integrally formed.

As described above, the inner lining 612 of the bite plate 610 may receive the teeth of one of the upper jaw or the lower jaw, and the inner lining 652 of the articulation plate 650 may receive the teeth of the other of the upper jaw or the lower jaw. Coupling of the bite plate 610 and the articulation plate 650 through engagement of the articulating components 622/662, posts 664 and housings 624 (as described in detail with respect to FIGS. 1A through 3C above) may maintain a proper positioning of the upper and lower jaw, and may thus reduce or substantially eliminate the deleterious effects of grinding, gnashing and/or clenching as described above.

In some implementations, one or more bite pads 670 may be positioned within the channel defined by the bite plate 610 and/or the articulation plate 650. For example, in some implementations, one or more bite pads 670 may be positioned in the channel, on the inner lining 612 of the bite plate 610 and/or the inner lining 652 of the articulation plate 650. The bite pads 670 may, for example, customize a fit of the bite plate 610 and/or the articulation plate 650, to provide cushioning for additional comfort during wear, and the like. In some implementations, the bite pads 670 may be removable and replaceable by the user to maintain proper fit, user comfort, good hygiene and the like. For example, regular removal of the bite pads 670 from the bite plate 610 and/or the articulation plate 650, and replacement with new bite pads 670, may enhance oral hygiene, and removal and replacement prior to excessive wear may maintain a proper amount of cushioning and a proper fit, thus enhancing effectiveness of the bruxism device. In some implementations, the bite pads 670 may be made of a relatively soft, relatively resilient thermoplastic material such as, but not limited to, plastic, or a material having properties that allow the bite pads 670 to conform to the teeth while also cushioning the jaw against impact and teeth-gnashing and clenching forces.

As shown in FIGS. 6A-6C, in some implementations, bite pads 670 may be positioned in left and/or right lateral portions of the bite plate 610 and/or the articulation plate 650. As shown in FIG. 6D, in some implementations, a bite pad 670 may be positioned in an anterior end portion of the first plate 610 and/or the second plate 650. As shown in FIG. 6E, in some implementations, a bite pad 670 may be U-shaped, following the contour of the bite plate 610 and/or the articulation plate 650, extending from a left rear portion, through the left lateral portion, the central anterior portion and the right lateral portion, to a right rear portion of the bite plate 610 and/or the articulation plate 650. In some implementations, a bite pad 670 positioned as shown in FIG. 6D and/or FIG. 6E may be positioned between a bumper portion of a damper 656 provided in the bite plate 610 and/or the articulation plate 650, to provide for cushioning between the bumper portion of the damper 656 and teeth received in the bite plate 610 and/or articulation plate 650.

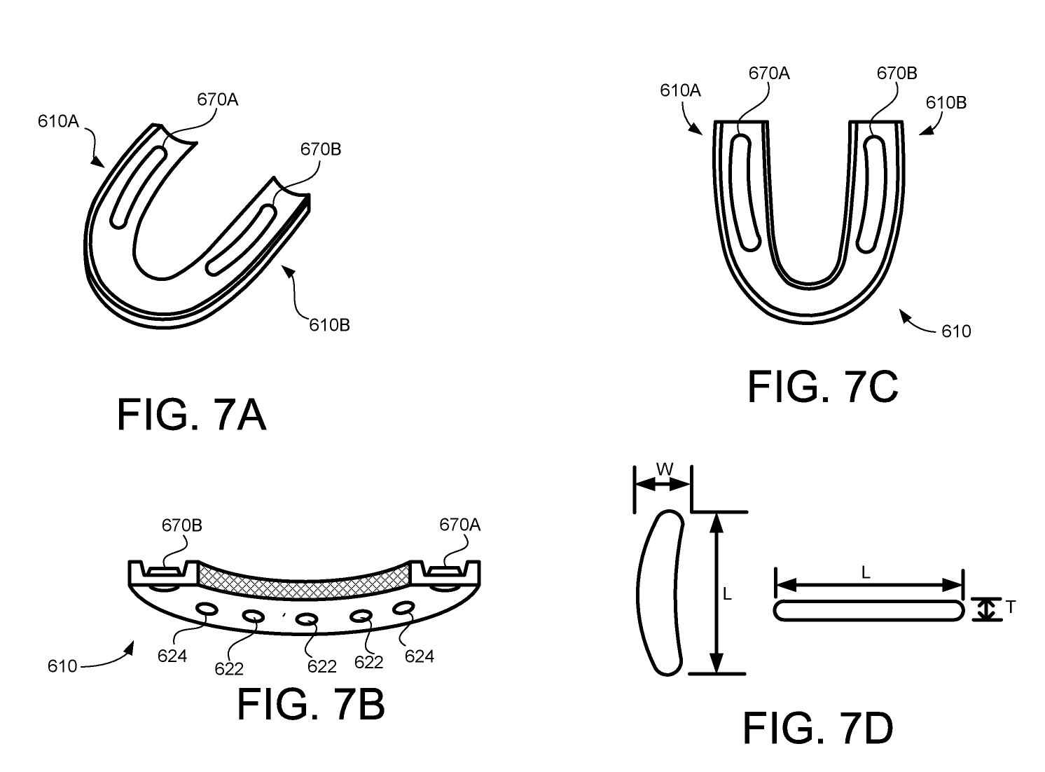

In some implementations, at least a portion of the bite pads 670 may have a somewhat curved, or arcuate, shape. For example, in some embodiments, a contour of the bite pads 670 may correspond to a contour of the portion of the channel defined by the inner portion of the bite plate 610 and/or articulation plate 650 in which the bite pads 670 are received, or the inner lining 612/652 in which the bite pads 670 are received, so that the bite pads 670 may be accurately positioned on the inner lining 612/652, between the inner lining 612/652 and the corresponding teeth. The example pair of bite pads 670 shown in FIG. 7A-7D may be positioned on the inner lining 612 of the bite plate 610, and/or on the inner lining 652 of the articulation plate 650.

Simply for ease of discussion and illustration hereinafter, the bite pads 670 shown in FIGS. 7A-7D will be described with respect to positioning of the bite pads 670 on the bite plate 610 as shown in FIG. 6A, with the bite plate 610 being positioned on the upper jaw and the inner lining 612 of the bite plate 610 receiving therein the teeth of the upper jaw. However, the example bite pads 670 shown in FIGS. 7A-7D may also be positioned on the bite plate 610 with the bite plate 610 being positioned on the lower jaw, and/or may be positioned on the articulation plate 650 with the articulation plate 650 being positioned on the upper jaw or the lower jaw. Similarly, the configuration of the bite pads 670 shown in FIGS. 7A-7D is simply one example implementation, and the bite pads 670 may have numerous other shapes, contours and the like, such as, for example, the bite pads 670 shown in FIGS. 6D and 6E.

A first bite pad 670A may be positioned in the channel defined in the inner portion of the bite plate 610, or on the inner lining 612, at a first lateral section 610A of the bite plate 610, for example a right lateral section 610A of the bite plate 610. A second bite pad 670B may be positioned in the channel defined in the inner portion of the bite plate 610, or on the inner lining 612 at a second lateral section 610B of the bite plate 610, for example a left lateral section 610B of the bite plate 610. In some implementations, the first bite pad 670A may be positioned so as to contact the teeth received in a rear portion of the first (right) lateral section 610A of the bite plate 610, and the second bite pad 670B may be positioned so as to contact the teeth received in a rear portion of the second (left) lateral side 610B of the bite plate 610. In some implementations, the right bite pad 670A and the left bite pad 670B may be interchangeable, depending on a method of attaching the bite pad 670 to the inner lining 612 and detaching the bite pad 670 from the inner lining 612. As shown in FIGS. 7B and 7C, in some implementations, a contour of the first bite pad 670A may correspond to a contour of the inner walls of the inner lining 612 of the first (right) lateral section 610A of the bite plate 610, and a contour of the second bite pad 670B may correspond to a contour of the inner walls of the inner lining 612 of the second (left) lateral section 610B of the bite plate 610. As noted above, in some implementations, a set of first and second bite pads 670A and 670B may be positioned in the channel defined in the inner portion of the bite plate 610, or on the inner lining 612 of the bite plate 610, as shown in FIGS. 7A-7C. In some implementations, another set of first and second bite pads 670A and 670B may be positioned in the channel defined in the inner portion of the articulation plate 650, or on the inner lining 652 of the articulation plate 650. In some implementations, a single bite pad may be positioned in the channel defined in the inner portion of the bite plate 610 and/or the articulation plate 650, and may extend from the rear portion of the first lateral section, through the anterior portion, and to the rear portion of the second lateral section of the bite plate 610/and or the articulation plate 650. In this case, the single bite pad may be somewhat ‘U’ shaped, to correspond to the shape of the channel formed in the inner portion of the bite plate and/or the articulation plate 650.

As shown in FIG. 7C, in some implementations, each bite pad 670 may extend from approximately 2 mm (posterior) to approximately 15 mm (anterior). In some implementations, a gap g of approximately 1 mm between a lateral edge of each bite pad 670 and a corresponding side wall of the inner lining 612 may be approximately 1 mm, with a space s between a posterior end of the bite pad 670 and a posterior end of the inner lining 612 of the bite plate 610 being approximately 2 mm. These dimensions represent just one example of the positioning and sizing of the bite pads 670 with respect to the plates 610/650. In some implementations, some, or all, of the dimensions referenced above may be greater than the stated approximate dimension. In some implementations, some, or all, of the dimensions referenced above may be less than the stated approximate dimension. The dimensions may vary based on numerous different factors, such as, for example, size and contour of the bite plate 610 and/or the articulation plate 650, specific user needs and preferences, and the like.

A size of the bite pad 670 may vary depending on various factors, such as, for example, a size of a particular bite plate 610 and/or articulation plate 650 into which the bite pad 670 is to be received, the size of the bite plate 610 and/or articulation plate 650 being based on a size of a mouth of a particular user. Bite pads 670 may be available in a variety of different sizes such as, for example youth sizes small, medium and large, adult sizes small, medium, large, extra-large and the like. Similarly, thickness of the bite pads 670 may vary based on various factors, such as, for example a length and width (i.e., size) of the bite pad 670, a level of cushioning and/or resilience desired by the user, fit and adjustment requirements of a particular user, and other such factors.

As shown in FIG. 7D, in some implementations, the bite pads 670 may have one or more curved portions. In some implementations, the bite pads 670 may have a somewhat ‘J’ shaped, or hooked, contour. In some implementations, the bite pads 67 may have a combination of curved portions and straight portions. In some implementations, a ratio of a length L of the bite pad 670 to a width W of the bite pad 670 may be approximately 2:1. In some implementations, a ratio of the length L to a thickness T of the bite pad 670 may be approximately 4:1. In some implementations, a ratio of the width W to the thickness T of the bite pad 670 may be approximately 2:1. These ratios represent just one example of the relative sizing of the bite pads 670. In some implementations, some, or all, of the ratios referenced above may be greater than the stated approximate ratio. In some implementations, some, or all, of the ratios referenced above may be less than the stated approximate ratio. The ratios may vary based on numerous different factors, such as, for example, size and contour of the bite plate 610 and/or the articulation plate 650, specific user needs and preferences, and the like.

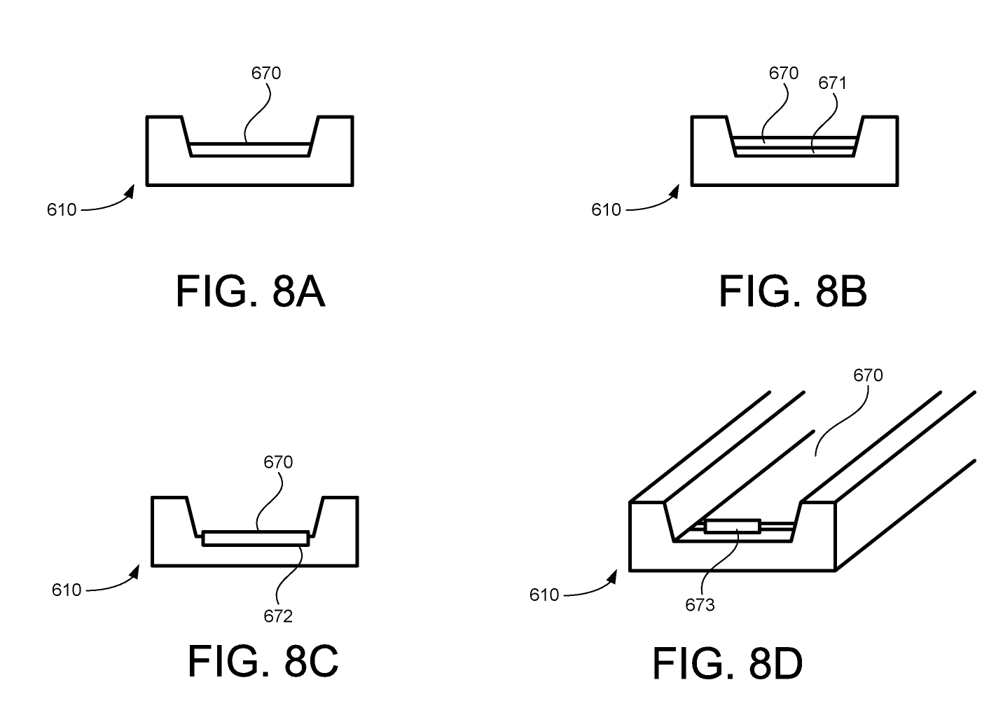

The bite pads 670 may be removably coupled to the bite plates by, for example, a coupling member. For example, in some implementations, the bite pads 670 may be, for example, friction coupled to the channel defined in the inner portion of the bite plate 610 by friction between the bite pads 670 and the side walls of the channel, or may be friction coupled to the inner lining 612 by friction between the bite pads 670 and the inner lining 612, as shown in FIG. 8A. In some implementations, the bite pads 670 may be coupled to the inner lining 612 by, for example, an adhesive 671 that temporarily bonds the bite pads 670 to the channel or to the inner lining 612, as shown in FIG. 8B. In some implementations, the bite pads 670 may be positioned in a recessed portion 672 of the channel, or the inner lining 612, to properly align and maintain a position of the bite pad 670, as shown in FIG. 8C. In some implementations, one or more ridges 673, or stops 673 may extend upward from a base wall 674 of the channel or the inner lining 612, as shown in FIG. 8D, for example, to establish a position of an anterior end and/or a posterior end of the bite pad 670 in the channel or the inner lining 612. The bite pads 670 may be positioned against the ridge(s) 673 to establish and maintain a position of the bite pads 670.

In some implementations, the bite pads 670 may be made of, for example, a thermoplastic elastomer material. In some embodiments, the material of the bite pads 670 may include properties which inhibit microbial growth. The bite pads 670 may vary in a degree of hardness and/or softness, depending on user preferences and/or oral/dental needs of a particular user.

In some implementations, the bite pads 670 may include a flavoring and/or scent. The flavoring may enhance user comfort, making the bruxism device more palatable for extended wear. The scent may provide a freshening effect during extended wear. In some embodiments, the flavoring and/or scent may be infused in the material of the bite pads 670, and may be released in response to pressure applied to the bite pads 670 by the teeth. The flavoring and/or scent may include, for example, a mint flavor and/or scent, a fruit flavor and/or scent, and the like.

In some implementations, one or more of the outer surfaces of the bite pads 670 may be patterned or textured, to facilitate and maintain placement of the bite pads 670 in the bite plate 610 and/or the articulation plate 650.

As noted above, the timely, regular removal and replacement of bite pads 670 in the bite plate 610 and/or the articulation plate 650 may improve oral hygiene. The timely, regular removal and replacement of bite pads 670 in the bite plate 610 and/or the articulation plate 650 may also maintain proper cushioning and help maintain proper positioning of the teeth in the bite plate 610 and/or the articulation plate 650, thus further facilitating the dissipation of forces associated with grinding and/or gnashing and/or clenching of the teeth and jaws and enhancing the effectiveness of the bruxism device.

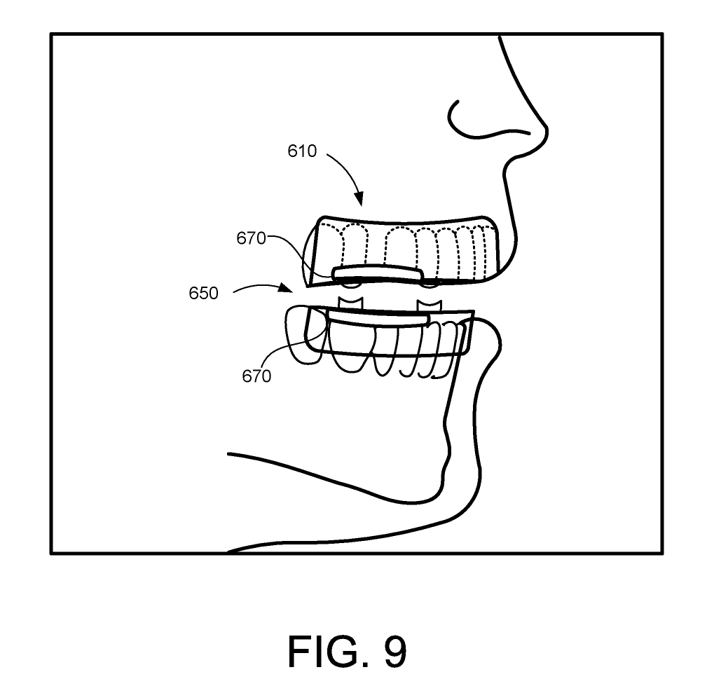

In use, the oral device 600 including the bite plates 670 may be worn by the user as shown in FIG. 9A, with the bite plate 610 mounted on the upper jaw and the articulation plate 650 mounted on the lower jaw. The upper bite pads 670 may be positioned between the inner lining 612 of the bite plate 610 and the teeth of the upper jaw, and the lower bite pads 670 may be positioned between the inner lining 652 of the articulation plate 650 and the teeth of the lower jaw. As described above, the bite plate 610 may be mounted on the lower jaw, and the articulation plate 650 may be mounted on the upper jaw, with the upper bite pads 670 positioned between the inner lining 652 of the articulation plate 650 and the teeth of the upper jaw, and the lower bite pads 670 be positioned between the inner lining 612 of the bite plate 610 and the teeth of the lower jaw.

The foregoing provides a detailed description of various exemplary embodiments. It is understood that modifications may be made without departing from the spirit and scope as broadly described herein and as set forth in the following claims.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. An oral device, comprising:
a first plate defining a first channel configured to receive teeth of one of an upper jaw or a lower jaw;
a second plate defining a second channel configured to receive teeth of the other of the upper jaw or the lower jaw;
a coupling device configured to couple the first plate and the second plate;
an articulating coupler configured to allow for articulation between the first plate and the second plate coupled by the coupling device; and
a plurality of bite pads configured to be removably coupled in at least one of the first channel defined in the first plate or the second channel defined in the second plate.

2. The device of claim 1, wherein the first channel extends along a contour of the first plate, the first channel being defined by a base wall, a first side wall extending from a first side of the base wall, and a second side wall extending from a second side of the base wall opposite the first side of the base wall.

3. The device of claim 2, wherein the contour of the first plate includes:
a first lateral section extending from a first posterior portion of the respective plate to a first anterior portion of the first plate;
a second lateral section extending from a second posterior end of the respective plate to a second anterior portion of the first plate; and
a curved section connecting an anterior end of the first lateral section and an anterior end of the second lateral section.

4. The device of claim 3, wherein the plurality of bite pads includes:
a first bite pad positioned on a first portion of the base wall corresponding to the first lateral section; and
a second bite pad positioned on a second portion of the base wall corresponding to the second lateral section.

5. The device of claim 4, wherein a shape of the a first bite pad corresponds a shape of the first portion of the base wall corresponding to the first lateral section, and a shape of the second bite pad corresponds to a shape of the second portion of the base wall corresponding to the second lateral section.

6. The device of claim 4, further comprising a first coupling member coupling the first bite pad to the first portion of the base wall and a second coupling member coupling the second bite pad to the second portion of the base wall.

7. The device of claim 1, wherein the first plate is a bite plate including an first inner liner received in a rigid first outer liner, the first channel being defined in the first inner liner so as to correspond to the teeth of the one of the upper jaw or the lower jaw to be received therein, and
the second plate is an articulation plate including a second inner liner received in a rigid second outer liner, the second channel being defined in the second inner liner so as to correspond to the teeth of the other of the upper jaw or the lower jaw to be received therein.

8. The device of claim 7, wherein the plurality of bite pads are positioned on at least one of
a base wall of the first channel so as to be positioned between the first inner liner and the teeth of the one of the upper jaw or the lower jaw to be received therein, or
a base wall of the second channel so as to be positioned between the second inner liner and the teeth of the other of the upper jaw or the lower jaw to be received therein.

9. The device of claim 1, wherein the articulating coupler includes:
a plurality of first articulating components included on the first plate, each of the first articulating components including a protrusion having a substantially semispherical shape extending outward from a mating surface of the first plate toward a mating surface of the second plate in the coupled state of the first and second plates; and
a plurality of second articulating components included on the second plate, each of the second articulating components including a receiving surface having a concave curved contour so as to interact with the semispherical surface of the first articulating component and guide a 360 relative motion between the first and second plates.

10. The device of claim 1, wherein the coupling device includes:
a plurality of posts extending outward from and substantially perpendicular to a mating surface of the second plate facing the first plate; and
a corresponding plurality of housings defined by a plurality of recesses in a mating surface of the first plate facing the second plate and configured to respectively receive the plurality of posts so as to couple the first and second plates.

11. The device of claim 10, wherein a distal end of each of the plurality of posts is fixed in a respective one of the plurality of housings in the first plate, and a cross sectional shape of each of the plurality of posts corresponding to a cross sectional shape of the respective housing in which it is received, each of the plurality of posts being solid and made of a flexible material so as to allow for six degree of freedom movement between the first and second plates within a range of motion of the plurality of posts.

12. The device of claim 1, further comprising a damper included in one of the first plate or the second plate, wherein the damper includes a cam provided in the one of the first plate or the second plate, the cam being configured to impart a separating force on a mating surface of the one of the first plate or the second plate in response to an external actuation force applied to the damper, the separating force increasing a distance between the mating surface of the one of the first plate or the second plate and at least one tooth received therein.

13. An oral device, comprising:
a bite plate configured to receive teeth of one of an upper jaw or a lower jaw therein;
an articulation plate configured to receive teeth of the other of the upper jaw or the lower jaw therein;
a coupling device configured to couple the articulation plate and the bite plate;
an articulation coupler configured to allow for articulation between the bite plate and the articulation plate coupled by the coupling device; and
a plurality of bite pads configured to be disposed on an interior facing surface of the bite plate, between the bite plate and the teeth to be received therein, and on an interior facing surface of the articulation plate, between the articulation plate and the teeth to be received therein.

14. The device of claim 13, wherein the bite plate includes a rigid outer liner and an inner liner in the rigid outer liner, the inner liner of the bite plate configured to receive the teeth of the one of the upper jaw or the lower jaw, and the articulation plate includes a rigid outer liner and an inner liner in the rigid outer liner, the inner liner of the articulation plate configured to receive the teeth of the other of the upper jaw or the lower jaw.

15. The device of claim 14, wherein the plurality of bite pads includes:
a first bite pad removably coupled to a base wall of the bite plate, at a right lateral section of the inner liner of the bite plate, and a second bite pad removably coupled to the base wall of the bite plate, at a left lateral section of the inner liner of the bite plate; and
a third bite pad removably coupled to a base wall of the articulation plate, at a right lateral section of the inner liner of the articulation plate, and a fourth bite pad removably coupled to the base wall of the articulation plate, at a left lateral section of the inner liner of the articulation plate.

16. The device of claim 15, wherein the first bite pad and second bite pad are to be positioned between the inner liner of the bite plate and the teeth to be received in the bite plate, and the third bite pad and the fourth bite pad are to be positioned between the inner liner of the articulation plate and the teeth to be received in the articulation plate.

17. The device of claim 15, further comprising a first coupling member coupling the first bite pad and the second bite pad to the inner liner of the bite plate, and a second coupling member coupling the third bite pad and the fourth bite pad to the inner liner of the articulation plate.

18. The device of claim 15, wherein a shape of the a first bite pad corresponds to a contour of the right lateral section of the inner liner of the bite plate, a shape of the second bite pad corresponds to a contour of the left lateral section of the inner liner of the bite plate, a shape of the third bite pad corresponds to a contour of the right lateral section of the inner liner of the articulation plate, and a shape of the fourth bite pad corresponds to a contour of the left lateral section of the inner liner of the articulation plate.

19. The device of claim 13, wherein the coupling device includes:
at least one post defined by a solid protrusion extending outward from a mating surface of the articulation plate; and
at least one housing defined by a recess in a mating surface of the bite plate, a cross section of the at least one post corresponding to a cross section of the at least one housing to fix a distal end of the at least one post in the at least one housing and couple the articulation plate and the bite plate, and
wherein the articulation coupler includes:
at least one first articulating component included on the mating surface of the bite plate, including a semispherical protrusion that extends outward from the mating surface of the bite plate; and
at least one second articulating component included on the mating surface of the articulation plate, including a receiving surface that engages the semispherical protrusion of the at least one first articulating component, a contour of the receiving surface being defined by a concave curved shape in which the semispherical protrusion of the at least one first articulating component is received.

20. An oral device, comprising:
a first plate configured to receive teeth of an upper jaw therein;
a second plate configured to receive teeth of a lower jaw therein;
a plurality of bite pads removably coupled to at least one of the first plate or the second plate, the plurality of bite pads being positioned between the respective plate and teeth received therein.
